(19) United States
(12) Patent Application Publication
(10) Pub. No.: US 2009/0247834 A1
(43) Pub. Date: Oct. 1, 2009

(54) QUALITY OF LIFE MANAGEMENT PROGRAM

(76) Inventor: Alan M. Schechter, Long Beach, CA (US)

Correspondence Address: STETINA BRUNDA GARRED & BRUCKER, 75 ENTERPRISE, SUITE 250, ALISO VIEJO, CA, 92656, US

(21) Appl. No.: 12/057,749

(22) Filed: Mar. 28, 2008

(51) Int. Cl. A61B 5/00

(52) U.S. Cl. 600/300

(57) ABSTRACT

The present invention is directed to a method for improving the quality of life of a patient. The method involves assessing the patient's quality of life by evaluating parameters relating to the patient's health, and assigning a score in relation to the evaluated parameters. In one version, the patient's score is compared to a standard score, and a treatment program is assigned to the patient on the basis of the comparison. The evaluated parameters may be related to the patient's mental health and physical health, and a mental health score and a physical health score can be assigned. In another version, the mental health score is compared to the physical health score, and a treatment regimen is assigned on the basis of the comparison. The method allows for improvement in quality of life of the patient by providing for a substantially objective assessment of parameters relating to the patient's health.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

1. Technical Field

The present invention relates generally to a program and method for the improvement in quality of life of a person suffering from at least one of mental and/or physical illness, disease, trauma, debilitating injury, or other condition, such as a person suffering from a chronic illness.

2. Related Art

The current state of the art in medicine and medical care is highly effective in the treatment of physical symptoms of illness and disease. However, modern medical methods used to assess such physical symptoms are often not adequate to properly evaluate the ability of the patient to adequately perform day-to-day tasks, or to evaluate the person's mental health state, which are factors that can profoundly affect the patient's overall quality of life. The improvement and maintenance of the patient's quality of life can be especially important for those patients suffering from terminal or chronic illness, where the physical prognosis of the patient is not expected to substantially improve. In these cases, maintaining the patient's quality of life, including maintaining a sense of optimism and personal satisfaction in life, as well as the ability to interact with others in social settings and perform day-to-day tasks, becomes of the utmost importance, and can even be more important than halting or slowing the progression of disease. For example, in terminal cancer cases, many patients struggle to balance their need to maintain their psychological sense of well-being and satisfaction with life, against the potential to prolong their life span with aggressive treatments that may adversely induce added pain and suffering.

Quality of life can generally be defined as the degree of well-being felt by an individual or group of people, and can be understood to consist of two components, a physical component, which can be controlled by factors such as a patient's health and diet as well as by control of pain and progression of disease, as well as a psychological component, which is regulated by factors such as stress, anxiety, pleasure and other positive or negative emotional states. Other external factors that can affect quality of life can include financial status, housing, employment, spirituality, social support network, and health. However, the combination of attributes that leads one individual to be content is rarely the same for another, making it difficult to predict the actual quality of life being experienced by an individual based merely on external factors. Also, a patient's own expectations and ability to cope with limitations can greatly affect the person's perception of health and satisfaction with life, and thus two people with the same health status as measured by conventional diagnostic metrics may in fact be experiencing very different internal qualities of life.

The term “health-related quality of life” (HRQL) is often used to describe the quality of life as it is affected by health and health care. This HRQL is distinguished from the patient's “functional status,” which is a term used to describe the patient's ability to function in physical, social and emotional realms. The patient's functional status is a subset of the person's functional capacity, and will vary according to how closely the patient's daily performance approaches their maximal functional capacity. In other words, functional status reflects the objective ability of a patient to perform the tasks of daily life. In contrast, HRQL reflects the subjective experience of the impact of health status on the patient's quality of life.

Understanding quality of life is particularly important in health care today, as monetary or other measures often used to quantify medical success often do not correlate well with a patient's sense of well-being. Decisions on what research to perform, treatments to invest in, and programs to initiate or direct patients to are closely related to their effect on the patient's quality of life. In fact, one of the more important developments in health care in the past decade may be the recognition that the patient's perspective is as legitimate and valid as the clinician's in monitoring health care outcomes, as is discussed for example in the article “Outcomes Measurement: A Report from the Front” by Geigle et al., Inquiry 1990, 27:7-13, and the article “The Problem of Quality of Life in Medicine” by Leplege et al., JAMA 1997, 278:47-50, both of which are herein incorporated by reference in their entireties.

The added value provided in better understanding the impact of disease from the patient's perspective has led to the development of instruments to attempt to quantify the patient's perception of their health status before and after treatment. Such instruments seek to measure quality of life via means other than standard objective physiological testing, which typically gives little information about the impact of the condition or treatment from the patient's perspective. Various authors have sought to better define health-related quality of life (HRQL) and to develop metrics for its assessment. For example, HRQL has been defined as a measure of the patient's perspective representing the “functional effect of an illness and its consequent therapy upon a patient, as perceived by the patient, as described in “Quality of Life Studies: Definitions and Conceptual Issues” by Schipper et al., Quality of Life and Pharmacoeconomics in Clinical Trials [2^{nd} Edition] Edited by Spilker B. Philadelphia, Lippincott-Raven Publishers; 1996: 11-23, which is herein incorporated by reference in its entirety. An alternative definition gives HRQL as “the value assigned to duration of life as modified by the impairments, function states, perceptions, and social opportunities that are influenced by disease, injury, treatment or policy” in “Health Status and Health Policy. Quality of Life in Health Care Evaluation and Resource Allocation” by Patrick et al., New York, Oxford University Press; 1993: 1-478, which is herein incorporated by reference in its entirety. HRQL is considered an important outcome measure in investigations of therapeutic interventions for patient with chronic conditions such as cancer and heart disease, in epidemiological studies and in patient care, representing a paradigm shift in the assessment of efficacy and effectiveness of therapeutic treatments, as described in the article “Quality of Life and Clinical Trials,” Lancet 1995, 346: 1-2, which is herein incorporated by reference in its entirety.

However, a problem with the HRQL questionnaires that are in existence is that they are mainly intended for the purposes of evaluating clinical research, and as such do not provide any guidance or insight into how individual patients can be helped to improve their quality of life. For example, while such questionnaires can allow for the tracking and comparison of an individual patient's responses over time, there is currently no standard clinical methodology for objectively quantifying an individual person's quality of life based on such questionnaires. There are also no standard methodologies for using such information about a person's quality of life to modify and/or improve their treatment or sense of well-being and thereby improve the person's quality of life.

Accordingly, there remains a need for methodologies capable of providing a substantially objective measure of the quality of life of individual patients as it is perceived by those patients. There is also a need for methodologies capable of evaluating the patient's quality of life, and using such evaluations to determine treatments or other actions that can be taken to improve quality of life.

### BRIEF SUMMARY OF THE INVENTION

The present invention specifically addresses and alleviates, among other things, the above-identified deficiencies in the art. In this regard, the present invention is directed to a method for improving the quality of life of a patient. In one embodiment, the method involves assessing the patient's quality of life by evaluating parameters relating to the patient's health, and assigning a score in relation to the evaluated parameters. The patient's score is compared to a standard score, and a treatment regimen is assigned to the patient on the basis of the comparison. The method allows for a substantially objective determination of the health status of the patient, thereby allowing for improvement in the patient's quality of life via selection of treatment regimens that are best suited to the patient on the basis of the score comparison.

In another embodiment, the method for improving the quality of life of a patient involves evaluating parameters relating to the patient's mental health and physical health, and assigning a mental health score and a physical health score in relation to the evaluated parameters. The patient's mental health score is then compared to the patient's physical health score, and a treatment regimen is assigned to the patient on the basis of the comparison. One or more of the methods can also be performed with the assistance of a computer software program having program code operative to perform one or more of the assessment and evaluation, comparison, and treatment assignment steps.

The present invention is best understood by reference to the following detailed description when read in conjunction with the accompanying drawings.

### BRIEF DESCRIPTION OF THE DRAWINGS

These as well as other features of the present invention will become more apparent upon reference to the drawings wherein:

FIG. 1 is a flow chart diagram illustrating an embodiment of a method for improving a patient's quality of life according to the present invention, involving evaluating parameters relating to the patient's health and comparing to a standard; and

FIG. 2 is a flow chart diagram illustrating an embodiment of another method for improving a patient's quality of life according to the present invention, involving evaluating parameters relating to the patient's mental and physical health and comparing to one another.

Common reference numerals are used throughout the drawings and detailed description to indicate like elements.

### DETAILED DESCRIPTION OF THE INVENTION

The detailed description set forth below is intended as a description of the presently preferred embodiment of the invention, and is not intended to represent the only form in which the present invention may be performed or utilized. The description sets forth the functions and sequences of steps for performing and operating the invention. It is to be understood, however, that the same or equivalent functions and sequences may be accomplished by different embodiments and that they are also intended to be encompassed within the scope of the invention.

It has been discovered that the quality of life of a patient can be improved by methods involving the assessment of the patient's quality of life, and the assignment of a treatment regimen on the basis of the assessment. In particular, in one embodiment it has been found that the patient's quality of life can be assessed by evaluating parameters relating to the patient's health, and assigning a score in relation to the evaluated parameters, comparing the patient's score to a standard score, and assigning a treatment regimen to the patient on the basis of the comparison. In another embodiment, the method for improving the quality of life of the patient can comprise evaluating parameters relating to the patient's mental health and physical health, and assigning a mental health score and a physical health score in relation to the evaluated parameters, comparing the patients mental health score to the patient's physical health score, and assigning a treatment regimen to the patient on the basis of the comparison. The methods provide a substantially objective means of quantifying the patient's quality of life, and allow for the selection of treatment directed at improving the patient's quality of life based on the objective assessment. The assessment and evaluation of the patient's quality of life according to the methods described herein also allows for healthcare providers, insurance companies, managed care operations, disease management companies, and the patient's themselves to better determine the effective treatments and programs that improve or maintain the patient's quality of life when suffering from chronic and/or debilitating conditions.

The patient evaluated and treated by the method can be any patient being seen and/or treated in a clinical and/or medical environment, or a person contemplating such medical and/or therapeutic treatment, such as a person recently diagnosed with a condition. Suitable patients may in particular be those suffering from a condition that is at least one of chronic, long-term and debilitating illness and/or injury and disease. The patient's medical condition may be primarily physical or mental, or may have components of both. Examples of conditions from which the patient may be suffering include but are not limited to heart disease (e.g., congestive heart failure and/or chronic heart failure), cancers, diabetes, asthma, HIV/AIDS, multiple sclerosis, systemic lupus, cystic fibrosis, hemophilia, chronic obstructive pulmonary disease, Alzheimer's disease, Huntington's disease, schizophrenia, depression, obesity, osteoporosis, ischemic cardiopathy, cerebrovascular disease, rheumatoid arthritis, osteoarthritis, chronic renal failure and partial or complete paralysis. The method of the instant invention can be performed in a hospital and/or clinical setting with the assistance, or under the administration, of a physician or other medical care professional. The steps can also be individually performed in separate settings, such as by performing one or more steps in a first physician's office and/or at the patient's home, and performing one or more second steps in a hospital or clinical treatment environment.

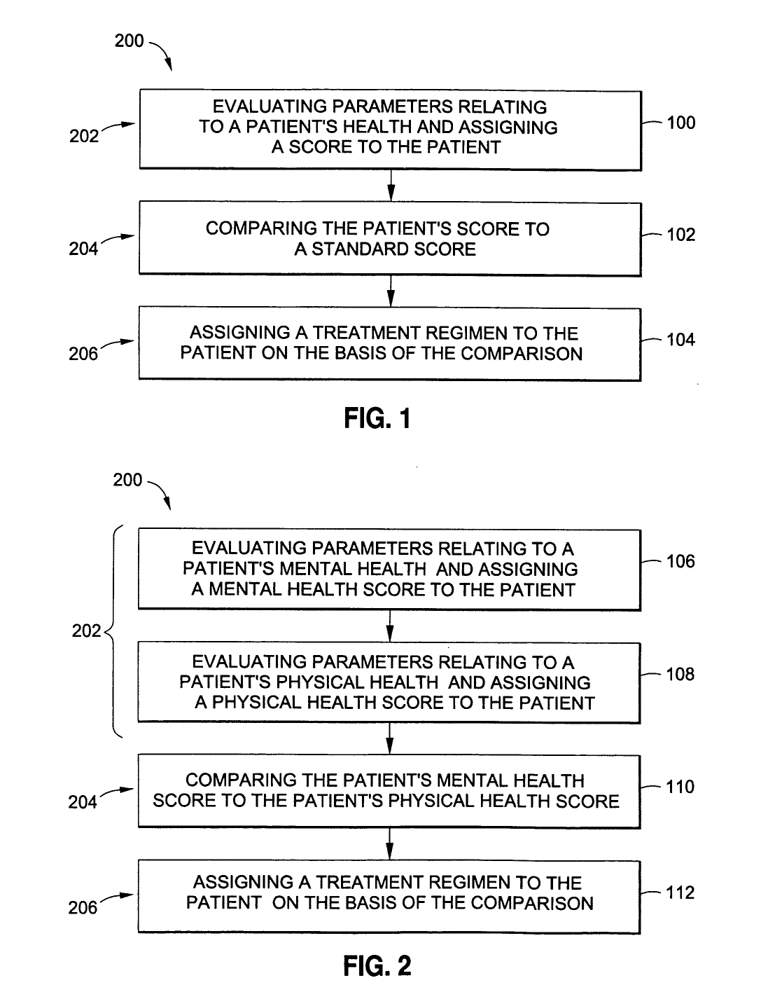

In one embodiment, as shown in FIG. 1, a first step 100 of the method involves assessing the patient's quality of life by evaluating parameters relating to the patient's health. The parameters can relate to one or more of physical and mental health parameters, and in particular may be those parameters that assist in quantifying the patient's overall quality of life. The parameters related to the patient's health can include but are not limited to aspects of the functional status and functional capacity of the patient, the ability of the patient to perform day-to-day tasks and engage in social activities, the level of pain or discomfort being experienced by the patient, the patient's level of anxiety, and the patient's general sense of well-being. Once these parameters have been evaluated, a score is assigned in relation to the evaluated parameters in order to assist in quantifying the patient's quality of life.

In one version, the parameters relating to the patient's health are assessed by providing at least one health evaluation questionnaire for the patient to complete. The health evaluation questionnaire may be a standardized form including various questions that are designed to evaluate one or more of the mental and physical health parameters. For example, the health evaluation questionnaire can comprise one or more questions relating to physical health parameters, mental health parameters, or a combination thereof. The health evaluation questionnaire is provided to the patient in paper or electronic form, and may also be completed with assistance from the patient's physician or other medical professional. In one version, the health evaluation questionnaire is available on-line at a health care provider's website or other website, and may also be available at a computer terminal located in a health care facility. Examples of standardized forms that can be used to evaluate parameters relating to the patient's health include various health-related quality of life (HRQL or HRQoL) surveys known in the art, such as the SF-36 questionnaire as described in the article “Health-Related Quality of Life in Urban Surgical Emergency Department Patients: Comparison with a Representative German Population Sample” by Neuner et al., Health and Quality of Life Outcomes 2005, 3:77, which is herein incorporated by reference in its entirety. By “completing” the questionnaire it is meant that the patient is given the opportunity to answer the questions thereon, and not necessarily that the patient has in fact answered every single question.

Examples of some questions that may be found on the health evaluation questionnaire include, but are not limited, to questions inquiring into: the patient's opinion of their current general health versus their opinion of their health a year ago; the amount of physical activity the patient is able to engage in on a regular basis; the ability of the patient to perform day-to-day tasks such as climbing stairs or carrying groceries; the amount of social interaction and/or productive work the patient engages in on a regular basis, and the level of any anxiety experienced during such social contact and/or work; general mood, sense of happiness and satisfaction felt by the patient; and any depression felt by the patient. Thus, the questions are preferably intended to not only gauge the patient's actual functional status, but are also intended to gauge the patient's perception of their own functional status and/or physical condition, which is indicative of the patient's overall sense of well-being. For example, the patient may be asked both whether they are able to climb a flight of stairs and/or perform work as well as whether their ability/inability to do so has adversely impacted them, and whether they perceive their current condition to be improved or worsened over their previous state.

Points are awarded according to the patient's answer for each question, and the points can be totaled to arrive at a score corresponding to the evaluated health parameters, as shown in step 100 of FIG. 1. The score may be cumulative of both mental and physical health parameters, or alternatively a physical health score can be assigned that corresponds to answers to health questions relating to physical health parameters, and a mental health score can be assigned that corresponds to answers to health questions relating to mental health parameters. The patient can also be assigned multiple scores corresponding to mental health, physical health and cumulative health scores. The points awarded to each question can also be weighted according to their relative importance to the results of the questionnaire. Health evaluation questionnaires directed to assessing status other than health-related quality of life (HRQL) can also be administered to the patient, such as questionnaires directed to evaluating the patient's diet and exercise, or evaluating the patient's compliance with recommended treatments. Furthermore, while the step 100 of assessing the patient's quality of life is described with particular reference to the completion of at least one health evaluation questionnaire, it should be understood that the parameters relating to the patient's health can also be evaluated by performing one or more medical diagnostic tests in addition, or as an alternative, to the health evaluation questionnaire.

Once the patient's quality of life has been assessed and a score has been assigned in relation to the evaluated parameters, the next step 102 in the method comprises comparing the patient's score to a standard score to provide a basis by which the patient's score can be quantitatively evaluated. In one version, the standard score corresponds to the mean score obtained from a group of persons and/or patients who have been evaluated and scored by the same or similar method as that used to evaluate and score the patient. For example, the standard score may correspond to the mean score obtained from a group of persons and/or patients that have completed the same or similar health evaluation questionnaire(s) completed by the patient. The standard score can comprise at least one of a standard mental health score and a standard physical health score, each of which corresponds to the mean mental and physical health scores, respectively, for the standard group of persons and/or patients. A comparison of the patient's score to the mean group score allows for the relative well-being of the patient as compared to the group to be objectively assessed. For example, the patient's physical health score can be compared to the average group physical health score to evaluate whether the patient's physical health is better or worse than the average group score. Similarly, the patient's mental health score and/or cumulative score can be compared to the average group score to evaluate whether the patient is doing better or worse than the group average. Thus, the comparison of the patient's score to the standard score allows for the patient's well-being to be substantially objectively quantified, thereby giving a more accurate assessment of the patient's condition as well as their perception thereof.

The group of persons on which the standard scores are based is selected according to the desired comparison to be made. In one version, the group of persons is selected to provide a comparison to other patients suffering from the same or similar conditions as the subject patient, and can even comprise patients in the same stage of illness as the subject patient. For example, for a patient suffering from cancer, the standard scores to which the patient is compared may be those for a group of patients also suffering from cancer in a similar stage of the disease to determine whether the patient is progressing as well as, better than, or worse than the average of patients suffering from a similar condition. In another version, the group of persons is selected to provide a comparison to other patients of the same age or to other patients within a predetermined age group range. As such, it is possible to determine whether the patient's well being is better than, worse than, or substantially similar to patients of the same age or age group. The group of persons on which the standard score is based may also be selected to allow for a comparison of a variety of other factors including but not limited to gender, health history, location, socioeconomic status, the type and duration of treatment being received, and the like. The standard scores may also be those corresponding to the general population, such as the average scores obtained from a large-scale survey of a regional or national population. Furthermore, the comparison of the patient's score is not required to be limited to only one standard group, but rather the patient's scores can be compared to a plurality of different standard scores obtained from different patient population groups, to provide a multi-dimensional analysis of the patient's treatment and overall condition.

The actual comparison of the patient's score to the standard score can involve a variety of different algorithmic manipulations performed to provide information regarding the state of the patient in relation to the state of the comparison group. In one version, the comparison of the patient's score to the standard score involves calculating the difference between the patient's score and the standard score, such as by subtracting the standard score from the patient's score. The difference in the score gives a measure of how much the patient deviates from the average of the comparison group. For example, at least one of the standard mental health, physical health and/or cumulative health scores may be subtracted from at least one of the patient's mental health, physical health and/or cumulative health scores, respectively. The difference in score can also be evaluated by subtracting the patient's score from the standard score, or by taking the absolute values of the calculated difference.

In yet another version, a standard deviation of the standard group score is obtained, such as by calculating the standard deviation of the mean group score according to conventional statistical methods. The calculated difference between the patient's score and the standard score is then compared to the standard deviation to determine whether it is statistically significant. For example, if the absolute value of the calculated difference is less than the absolute value of one standard deviation, then it may not be considered to be statistically significant, whereas if the absolute value of the calculated difference is greater than the absolute value of one standard deviation, then it may be considered to be statistically significant. The calculated differences and standard deviations can be compared for at least one of the patient's mental health score, physical health score and/or cumulative score, to provide an overall analysis of the patient's comparative quality of life.

The comparison of the difference between the patient's and standard score and the standard deviation can also be used to determine whether the patient's health status is average, better or worse than average as compared to the standard group. For example, if the patient's score is higher than the standard score, and the absolute value of the difference in scores exceeds the standard deviation of the standard score, then the patient's health status may be considered to be better than the average person in the comparison group. If the patient's score is higher than the standard score, but the absolute value of the difference in scores does not exceed the standard deviation of the standard score, then the patient's health status may be considered to be merely average as compared to the standard group. Conversely, if the patient's score is lower than the standard score, and the absolute value of the difference in scores exceeds the standard deviation of the standard score, then the patient's health status may be considered to be worse than the average as compared to the standard group. Finally, if the patient's score is lower than the standard score, but the absolute value of the difference in scores does not exceed the standard deviation of the standard score, then the patient's health status may also be considered to be merely average as compared to the standard group. Such comparisons can be performed to substantially objectively evaluate the patient's mental, physical and cumulative health with reference to the standard group. While the comparison being discussed herein is phrased in terms of the patient's score being “higher,” i.e., better than the standard score, or “lower,” i.e., worse than the standard score, it should be understood that the relation of the patient's score to the standard score will depend on the type of health evaluation questionnaire administered. For example, for questionnaires in which higher point values are awarded for answers indicative of good health status, a patient's score that is higher than the standard score may be indicative of better health. However, for questionnaires in which lower point values are awarded for answers indicative of good health status, a patient's score that is lower than the standard score may actually be indicative of better health.

In yet another version, the standard score to which the patient's score is compared can correspond to a ratings system for evaluating the patient's overall physical and mental health. For example, the standard score may comprise ranges within which the patient's health is ranked, such as ranges corresponding to excellent health, good health, average health, poor health and extremely poor health. The patient's score is compared to the standard score to determine which range the patient's score falls within, and the person is then assigned a health assessment in relation to the standard score range. The ratings system may be developed, for example, by determining ranges of scores expected for each health status based on questions presented in the health evaluation questionnaire. For example, for health evaluation questionnaires having high point values awarded for answers indicative of good health status, the ratings system may set a range of higher scores corresponding to good or excellent health, and a range of lower scores corresponding to poor or extremely poor health. The ratings system can comprise ratings for mental health scores, physical health scores, and/or cumulative health scores.

As a final step 104, a treatment regimen is assigned to the patient on the basis of the comparison between the patient's score and the standard score. For example, if it is determined that the patient's mental health score is indicative of a need for mental health treatment, such as by being below a standard deviation of the standard mental health score or by corresponding to a standard score in a rating system indicative of a need for treatment, then a treatment regimen may be proposed to the patient that is devised to improve the patient's mental health. As another example, if it is determined that the patient's physical health score is indicative of a need for physical health treatment, such as by being below a standard deviation of the standard physical health score or by corresponding to a standard score in a rating system indicative of a need for treatment, then a treatment regimen may be proposed to the patient that is devised to improve the patient's physical health. If both of the patient's mental and physical health scores are such that they indicate a need for treatment, then both mental and physical health regimens may be assigned, as appropriate. Alternatively, if the patient's scores are indicative that the patient's mental and/or physical health are better than the standard score, then such scores provide confirmation that the patient may be maintained on the same, apparently successful, treatment regimen.

The treatment regimen is assigned according to the particular condition and needs of the patient, as assessed by the comparative evaluation, and may comprise aspects of any available treatment suitable for the patient's particular medical condition. Examples of treatment regimens that can be assigned where the patient is in need of physical health improvement can include, but are not limited to, dietary programs, exercise programs, one or more courses of medication indicated for the treatment of the patient's condition and/or pain relief, surgical treatment, radiation therapy, physical therapy programs, check-ups and diagnostic testing by physicians, and outpatient and/or residential care programs, as well as combinations thereof Examples of treatment regimens that can be assigned where the patient is in need of mental health improvement can include, but are not limited to, individual or group psychotherapy, support programs, one or more courses of anxiety relieving and/or anti-depression medications, physical therapy and holistic treatment programs.

Aspects of the treatment regimen can also be directed to treatment of both mental and physical health states, such as by tailoring the treatment regimen to provide the necessary physical and/or mental health treatment. For example, for those patients progressing well physically, but doing poorly mentally, the patient's treatment regimen may be modified or newly assigned to provide more mental health treatment while de-emphasizing or maintaining existing physical health treatment, in proportion to the extent to which the patient's physical health exceeds their mental health. Conversely, for those patients doing well mentally but progressing poorly physically, the patient's treatment regimen may be modified or newly assigned to provide more physical health treatment while de-emphasizing or maintaining existing mental health treatment, in proportion to the extent to which the patient's mental health exceeds their physical health.

In one version, the treatment regimen assigned to the patient on the basis of comparison to a standard health score can comprise enrollment in a disease management program. Disease management programs involve clinicians and others responsible for the systematic treatment of patients and providing of patient care, involving evidence-based standards or guidelines for care, trained health care personnel, and monitoring of patients and health care costs. Some examples of disease management programs include, but are not limited to those focused on diabetes, asthma, heart disease (especially congestive heart failure), HIV/AIDS, multiple sclerosis, systemic lupus, cystic fibrosis and hemophilia. Diseases that are good candidates for disease management may be those having: (1) high aggregate costs, (2) a large portion of the costs attributable to drug therapy, (3) measurable health outcomes, (4) potential for short-term gains in health outcomes and cost savings, and (5) an otherwise large variation in treatment practice. Disease management programs may provide. (1) continuous care delivery systems that coordinate caregivers, (2) an integrated information base of clinical guidelines or protocols and patient information that is accessible to caregivers and patients, (3) an information base for the economic structure of the disease, (4) shifting of some chronic disease care from physicians to monitoring and care by patient themselves, (5) emphasis on educating patients on the importance and key aspects of self-care, and/or (6) a quality improvement system that feeds experience back into clinical and economic information bases. Disease management also supports patient centeredness, a key element in quality enhancement, by contributing to the physician or practitioner/patient relationship and plan of care. Disease management also emphasizes the prevention of exacerbations and complications utilizing evidence-based practice guidelines and patient empowerment strategies, and evaluates clinical, humanistic, and economic outcomes on an ongoing basis with the result of improving overall health and quality of life for patients and their families. Thus, disease management programs may be suitable for certain conditions in which the patient's mental and/or physical health status is in need of improvement. An exemplary disease management program is described in detail in U.S. patent application Ser. No. 11/514,585, the entirety of which is herein incorporated by reference.

In one version, a comparison of the patient's score to a standard score corresponding to an average for a group of patients receiving treatment in a disease management program may be used as the basis for determining whether to assign the patient to the disease management program. For example, if the patient has a statistically significant lower health score than other patients having the same or similar condition that are enrolled in the disease management program, then the patient may be a good candidate for the same disease management program. Alternatively, if the patient scores higher than other patients in a disease management program, then the patient may be maintained on the same course of treatment and not enrolled in the disease management program. Enrollment in a disease management program may be especially indicated for those patients having very low physical health scores as compared to patients that are enrolled in a disease management program, as such patients may benefit from the increased oversight and management provided by the programs. Also, those patients that score within an average range with regards to physical health in comparison to the patients enrolled in the disease management program, but score lower than the average with regards to mental health, may be good candidates for the disease management program. Such patients may find the increased support and health management provided by the disease management program to increase their sense of well-being and satisfaction and decrease their sense of anxiety about their condition and its management.

The progress of individual patients enrolled in the disease management program may also be monitored to determine whether the program is helping the patients. For example, if individual patients score very low on either mental or physical health parameters as compared to the group score, it may be indicative that the patient is in need of more highly individualized care, or should be re-assigned to a different disease management program that is more appropriate to the mental and/or physical health deficit with which the patient is coping. Conversely, if patients score higher or average as compared to the group score, then it may be appropriate to maintain them in the disease management program.

Yet another version of a method for improving the quality of life of a patient is shown in FIG. 2. In this method, a step 106 is performed in which parameters relating to the patients mental health are evaluated, and a mental health score is assigned in relation to the evaluated parameters. In another step 108, parameters relating to the patients physical health are evaluated, and a physical health score is assigned in relation to the evaluated parameters. These mental and physical health evaluation steps 106, 108 can be performed sequentially as shown or in reversed order, or may alternatively be performed substantially simultaneously. The mental and physical health parameters can be evaluated and the scores assigned according to any of the methods that have been previously described herein, such as via completion of a health evaluation questionnaire, or any of the other methods previously described in relation to FIG. 1. Once the scores have been assigned, a step 110 is performed to compare the patient's mental health score to the patient's physical health score, and in a final step 112 a treatment regimen is assigned to the patient on the basis of the score comparison.

Thus, in the embodiment of the method depicted in FIG. 2, a comparison of the patient's mental and/or physical health score to a standard score is not required, as the comparison of the patient's own scores provides information sufficient to inform the assignment of the treatment regimen. However, it should be noted that the various steps described in relation to FIGS. 1-2 can also be combined to formulate alternative methods. For example, a step 110 in which the patient's mental health score is compared to the patient's physical health score as depicted in FIG. 2 can also be combined with a method in which one or more of the scores are compared to a standard score, as in the method depicted in FIG. 1. As another example, a step 102 in which the patient's mental and/or physical health scores are compared to one or more standard scores as depicted in FIG. 1 can also be combined with a method in which such scores are compared to one another, as in the method depicted in FIG. 2.

In one version, the comparison of the patient's mental health score to the patient's physical health score takes the form of the evaluation of a ratio of the patient's physical health score to the patient's mental health score, or alternatively a ratio of the patient's mental health score to their physical health score, to determine therefrom which of the patient's physical or mental status is better. The patient's mental and/or physical health score can also optionally be weighted before this step is performed to provide the desired comparison. For example, one or more of the health scores can be weighted such that a hypothetical “equal” mental and physical health status would have the same numerical value. In this case, if the ratio of the patient's physical health score to mental health score is greater than one, then the patient is in greater need of mental health care than physical health care. If the ratio is less than one, then the patient is in greater need of physical health care than mental health care. If the ratio is substantially equal to one, then the patient has an equal need for mental and physical health care.

Accordingly, the assignment of a treatment regimen to a patient thus evaluated may proceed on the basis of determining whether the patient is in greater need of improvement in mental or physical health status. For example, when the patient's mental health is worse than the patient's physical health, as evidenced from a comparison of the patient's mental and physical health scores, then the patient may be assigned a new or modified treatment regimen directed towards improving the patient's mental health. Conversely, when the patient's physical health is worse than the patient's mental health, as evidenced from a comparison of the patient's mental and physical health scores, then the patient may be assigned a new or modified treatment regimen directed towards improving the patient's physical health. The treatment regimen assigned to the patient may be any of those previously described herein as being suitable to improve mental and/or physical health, such as those methods described in relation to FIG. 1. The patient may also be deemed a good candidate for enrollment in a disease management program, such as any of those described above, if the comparison reveals that the patient's mental health is substantially worse than the patient's physical health, as the support and health oversight and management of such programs may improve the patient's sense of well-being.

In general, the steps of the methods as shown in FIGS. 1-2 may also be repeated to continuously track and monitor patients receiving treatment for conditions such as chronic and/or debilitating illnesses or injury. As an example, a patient diagnosed with a chronic illness, such as diabetes mellitus, may be evaluated by one or more of the quality of life assessment methods described herein, and may be assigned a treatment regimen on the basis of the evaluation, such as enrollment in a disease management program that appears to be well suited based on the patient's physical and mental health needs. The patient may then be re-evaluated at subsequent intervals, such as once every six months or once a year, or subsequent to any modifications to the patient's treatment program, to determine whether the program is a good fit for the patient, or whether additional treatment and/or modifications are needed. The information provided by evaluating the patient has the added benefit of contributing data that can be used to formulate a new group average (e.g. standard score) that may serve as a standard basis for comparison for other patients in need of treatment.

In one version, the patient is continuously re-evaluated to determine whether the patient's course of treatment is suitable given their particular demographic circumstances and the circumstances of their condition. For example, the patient may be initially evaluated by comparison to one or more standard groups having the same and/or similar condition, which groups also share at least one, and preferably multiple other demographic and/or health factors with the patient, including but not limited to at least one of an age range, gender, geographic location, socioeconomic status, marital status, number of dependents, previous history of disease, stage of the condition and/or disease, medication being taken, religions affiliation, availability of home help, family health history, genetic pre-disposition, and any other demographic and/or health factors that may be of relevance in the treatment and progression of the condition. The one or more standard groups preferably comprise patients already enrolled in various types of treatment programs, such as disease management programs, which the patient and/or their physician may be considering as candidates for the treatment of the patient. For example, to compare programs for the treatment of cancer, the comparison groups may include patients receiving surgical treatment, radiation therapy, chemotherapy, or various combinations of such treatments, to provide a comparison to different treatment options. The comparison groups may also be formulated by evaluating segments of the patient populations enrolled in such treatment programs, where the segments correspond to those patients that also share one or more common demographic and/or health features with the subject patient. In this way, a multi-dimensional analysis may be performed to determine which particular type of treatment program may be best suited to the patient, not only on the basis of the patient's particular disease, but also on the basis of how well persons sharing common demographic and/or other features with the patient perform in quality of life assessments while enrolled in each treatment program.

The patient can be started in the treatment program that provides the best comparison to standard groups sharing common features with the patient. When the patient's demographic and/or health status changes, for example if the patient enters a new stage of illness, loses a spouse, re-locates, enters a new age group, etc., the patient can be re-evaluated with respect to standard groups sharing the new demographic and/or health factor(s). Thus, the continuous re-evaluation and comparison to standard groups sharing select factors allows for the personalized selection of treatment programs, thereby optimizing the treatment and care of the patient. It is important to note that since the comparison is based on an assessment of the patient's quality of life in comparison to the standard groups, the optimum treatment regimen is selected not only with regard to physiological metrics of success, but also with regard to how patients sharing similar traits and factors perceive their own health in the treatment regimen, thereby improving chances of selecting a program that will similarly foster an enhanced sense of well-being in the patient.

The assessment of the patient's quality of life can also be combined with other objective diagnostic metrics to provide improved treatment of the patient. For example, if the person's physical health score is determined to be low in comparison to the standard score, or is lower than the person's mental health score, yet the patient scores well in standard objective diagnostic metrics and physiological evaluations to test functional capacity, such as blood tests, assays, exercise tests, etc., it may be the case that the person's perception of their physical health is lower than it should be, or that the standard tests are not giving an adequate measure of the physical impact of the patient's condition on their day-to-day life. In such cases, treatment regimens targeted towards increasing the patient's perception and/or the reality of their physical health may be recommended, such as at least one of physical therapy, exercise programs, support groups, and the like.

In one embodiment, at least a portion of the above-described methods are performed via a computer software program 200 embedded in one or more computers, processing platforms and/or memory devices. The computer software program 200 may be written in any conventional software programming language, and may be compiled and/or executed on any conventional computer and/or processing platform known in the art, and may also be distributed over multiple processing platforms. The computer software program 200 comprises program code operative to implement steps of the above-described methods, such as assessment program code 202, comparison program code 204, and treatment program code 206, and thereby capable of evaluating and assigning treatment to patients to improve the patient's quality of life. In one version, the computer program 200 comprises or is incorporated into computer software used for a disease management program.

The assessment program code 202 comprises code that is operative to assess the patient's quality of life by evaluating the parameters relating to the patient's health, and assigning a score in relation to the evaluated parameters. For example, the assessment code 202 may evaluate parameters relating to the patient's mental health and assign a mental health score to the evaluated parameters, while also evaluating parameters relating to the patient's physical health and assigning a physical health score to the evaluated parameters. A cumulative health score may also be assigned by evaluating the health parameters. The assessment code 202 may perform the assessment and/or evaluation function, for example, by electronically administering a health survey questionnaire to a patient and scoring the questionnaire. The assessment code 202 may also be operative to receive an input corresponding to the answers submitted by the patient and/or their health care professional to such a questionnaire, and to total the points awarded to the questions to arrive at one or more of the mental, physical health and/or cumulative health scores.

The comparison program code 204 comprises code that is operative to compare the one or more scores obtained by the assessment program code 202 to substantially objectively quantify the patient's status. For example, in one version, the comparison program code 204 is operative to compare one or more of the scores assigned to the patient by the assessment program code 202 to one or more standard scores, such as a standard score corresponding to an average score for a select group of persons, or other standard score as has previously been described herein. The comparison program code 204 may comprise tables and/or databases of data including such standard scores, or may be capable of accessing remote databases containing such data. The comparison program code 204 may also be capable of determining which standard group to compare the patient's score to, such as by selecting groups in databases sharing one or more demographic, health or other features, and may even be capable of formulating such groups for comparison based on the shared features, using assessment and demographic data for different treatment groups stored in databases. The comparison program code 204 is also capable of performing one or more algorithmic manipulations to arrive at the comparison between the patient's score and standard score, such as evaluating a difference between the scores, evaluating a standard deviation of the standard score, and/or evaluating whether a difference between the scores has statistical significance, such as the algorithmic manipulations that have previously been described herein. In another version, the comparison program code 204 is operative to compare the patient's mental health score to their physical health score, such as by evaluating a ratio of the scores.

The treatment program code 206 is operative to assign a treatment regimen to the patient on the basis of the comparison obtained by the comparison software code 204. For example, the treatment program code 206 may contain or be capable of remotely accessing databases containing information on treatment regimens suitable for given conditions and their comparison values. The treatment program code 206 may also be capable of accessing databases having information on treatment regimens for one or multiple different conditions, thereby allowing for patients having multiple conditions to also be treated. The databases may contain, for example, treatment regimens indexed by the value of the comparison obtained by the comparison program code 204, as well as the type of condition from which the patient is suffering. For example, the treatment program code 206 may be capable of locating a recommended treatment regimen for a patient having a particular condition and exhibiting certain physical health and mental health scores, with either a particular difference between the scores and standard scores, or a particular ratio of the physical and mental health scores, as determined by the comparison program code 204. The treatment program code 206 may also be capable of assigning further diagnostic tests and/or appointments with physicians on the basis of the comparison, or may be capable of assigning dietary or physical exercise regimens. The treatment program code 206 may be further capable of referring the patient to a disease management program for enrollment or further evaluation. The treatment program code 206 may also be capable of notifying the patient's physician of the comparison, and receiving input from the physician that corresponds to the treatment regimen to be assigned to the patient. It should be understood that while the computer software program 200 is capable of performing any of the assessment and evaluation, comparison, and treatment assignment steps described herein, the program 200 is not limited to only those specific functions and operations particularly describe, but is also operative to perform other methods of evaluation, comparison and assignment of suitable treatment regimens not specifically described.

### EXAMPLES

The following examples illustrate embodiments of methods for improving the quality of life of a patient by performing steps to substantially objectively assess the patient's quality of life. It should be noted that the values presented herein have been rounded to their nearest value in the interests of clarity of the presentation.

Table 1 below provides physical health data obtained from ten patients in response to an HRQL questionnaire (health-related quality of life questionnaire.) The same questionnaire was also given to other patients making up a larger patient group. An objective assessment of the patients via algorithmic manipulation and/or comparison of the data was performed to determine the patient's physical status.

TABLE 1

| | | | | | | Physical | | |
|---|---|---|---|---|---|---|---|---|
| | Patient | Mean | | | | Health | Comp. | PCS |
| Patient # | PCS¹ | PCS² | Δ PCS³ | Δ PCS + 6.97⁴ | Δ PCS − 6.97⁴ | Status | PCS⁵ | Signif.⁶ |
| 1 | 38.26 | 33.40 | 4.86 | 11.83 | −2.11 | Extremely | Above | Not |
| | | | | | | Poor | Average | Significant |
| 2 | 30.63 | 33.40 | −2.77 | 4.20 | −9.74 | Extremely | Below | Not |
| | | | | | | Poor | Average | Significant |
| 3 | 51.86 | 33.40 | 18.46 | 25.43 | 11.49 | Very Poor | Above | Not |
| | | | | | | | Average | Significant |
| 4 | 36.42 | 33.40 | 3.02 | 9.99 | −3.95 | Extremely | Above | Not |
| | | | | | | Poor | Average | Significant |
| 5 | 43.72 | 33.40 | 10.32 | 17.29 | 3.35 | Seriously | Above | Not |
| | | | | | | Poor | Average | Significant |
| 6 | 42.15 | 33.40 | 8.75 | 15.72 | 1.78 | Seriously | Above | Not |
| | | | | | | Poor | Average | Significant |
| 7 | 36.23 | 33.40 | 2.83 | 9.80 | −4.14 | Extremely | Above | Not |
| | | | | | | Poor | Average | Significant |
| 8 | 23.55 | 33.40 | −9.85 | −2.88 | −16.82 | Extremely | Below | Significant |
| | | | | | | Poor | Average | |
| 9 | 41.36 | 33.40 | 7.96 | 14.93 | 0.99 | Seriously | Above | Not |
| | | | | | | Poor | Average | Significant |
| 10 | 13.78 | 33.40 | −19.62 | −12.65 | −26.59 | Extremely | Below | Significant |
| | | | | | | Poor | Average | |

¹Patient's Physical Component Score;
²Mean Physical Component Score of Group;
³Patient PCS minus Mean PCS;
⁴Standard Deviation of Mean PCS +/− 6.97;
⁵Comparative PCS;
⁶PCS Significance

As can be seen from Table 1, the patient's physical health status was assessed by comparing their Physical Component Score (PCS) to a standard ratings system, with most of the patients in this group exhibiting very poor to seriously poor health status. The PCS of each patient was then compared to the mean PCS of the entire group to determine whether each patient was below or above average in this group. Finally, the difference between each patient's PCS and the mean PCS was compared to the standard deviation of the mean PCS to determine whether the difference in the patient's PCS was statistically significant. In the ten patients shown, only patients 8 and 10 exhibited significant departures from the average PCS, even though three of the patients had PCS scores that were below average. Patients 8 and 10 are thus identified as good candidates for further treatment regimens directed toward improving physical health.

Table 2 below provides mental health data obtained from ten patients in response to an HRQL questionnaire (health-related quality of life questionnaire.) The same questionnaire was also given to other patients making up a larger patient group. An objective assessment of the patients via algorithmic manipulation and/or comparison of the data was performed to determine the patient's mental health status.

TABLE 2

| | | | | | | Mental | | |
|---|---|---|---|---|---|---|---|---|
| | Patient | Mean | Δ | | | Health | Comp. | MCS |
| Patient # | MCS¹ | MCS² | MCS³ | Δ MCS + 6.97⁴ | Δ MCS − 6.97⁴ | Status | MCS⁴ | Signif.⁵ |
| 1 | 32.80 | 47.36 | −14.56 | −7.59 | −21.53 | Extremely | Below | Significant |
| | | | | | | Poor | Average | |
| 2 | 48.01 | 47.36 | 0.65 | 7.62 | −6.32 | Seriously | Above | Not |
| | | | | | | Poor | Average | Significant |
| 3 | 55.67 | 47.36 | 8.31 | 15.28 | 1.34 | Very Poor | Above | Not |
| | | | | | | | Average | Significant |
| 4 | 55.33 | 47.36 | 7.97 | 14.94 | 1.00 | Very Poor | Above | Not |
| | | | | | | | Average | Significant |
| 5 | 57.62 | 47.36 | 10.26 | 17.23 | 3.29 | Very Poor | Above | Not |
| | | | | | | | Average | Significant |
| 6 | 43.36 | 47.36 | −4.00 | 2.97 | −10.97 | Seriously | Below | Not |
| | | | | | | Poor | Average | Significant |
| 7 | 35.60 | 47.36 | −11.76 | −4.79 | −18.73 | Extremely | Below | Significant |
| | | | | | | Poor | Average | |
| 8 | 60.07 | 47.36 | 12.71 | 19.68 | 5.74 | Poor | Above | Not |
| | | | | | | | Average | Significant |
| 9 | 53.91 | 47.36 | 6.55 | 13.52 | −0.42 | Very Poor | Above | Not |
| | | | | | | | Average | Significant |
| 10 | 70.08 | 47.36 | 22.72 | 29.69 | 15.75 | Average | Above | Not |
| | | | | | | | Average | Significant |

¹Patient's Mental Component Score;
²Mean Mental Component Score of Group;
³Patient MCS minus Mean MCS;
⁴Comparative MCS;
⁵MCS Significance

As can be seen from Table 2, the patient's mental health status was assessed by comparing their Mental Component Score (MCS) to a standard ratings system, with most of the patients in this group exhibiting very poor to seriously poor mental health status. The MCS of each patient was then compared to the mean MCS of the entire group to determine whether each patient was below or above average in this group. Finally, the difference between each patient's MCS and the mean MCS was compared to the standard deviation of the mean MCS to determine whether the difference in the patient's MCS was statistically significant. In the ten patients shown, only patients 1 and 7 exhibited significant departures from the average MCS, even though three of the patients had MCS scores that were below average. Patients 1 and 7 are thus identified as good candidates for further treatment regimens directed toward improving mental health.

Table 3 below provides a comparison of physical and mental health data obtained from the same ten patients in response to the HRQL questionnaire (health-related quality of life questionnaire) given to the patients. An objective assessment of the patients via algorithmic manipulation and/or comparison of the data was performed to determine the patient's overall health status.

TABLE 3

| | Ratio of Patient | |
|---|---|---|
| Patient # | PCS to MCS¹ | Overall Health Status |
| 1 | 1.17 | Physical Health is better than Mental |
| | | Health |
| 2 | 0.64 | Mental Health is better than Physical |
| | | Health |
| 3 | 0.93 | Mental Health is better than Physical |
| | | Health |
| 4 | 0.66 | Mental Health is better than Physical |
| | | Health |
| 5 | 0.76 | Mental Health is better than Physical |
| | | Health |
| 6 | 0.97 | Mental Health is better than Physical |
| | | Health |
| 7 | 1.02 | Physical Health is better than Mental |
| | | Health |
| 8 | 0.40 | Mental Health is better than Physical |
| | | Health |
| 9 | 0.77 | Mental Health is better than Physical |
| | | Health |
| 10 | 0.20 | Mental Health is better than Physical |
| | | Health |

¹Ratio of Patient's Physical Component Score to Patient's Mental Component Score

As can be seen from Table 3, the patient's Physical Component Score (PCS) and Mental Component Score (MCS) from Tables 1 and 2 above were used to calculate a comparative ratio of the scores. The patient's overall health was determined on the basis of the comparative ratios, with ratios greater than 1 being indicative of physical health status being better than mental health status, and ratios less than 1 being indicative of mental health status being better than physical health status. Most of the patients exhibited a mental health status that was better than their physical health status, with patients 1 and 7 differing in having a physical health status that was better than their mental health status. Patients 1 and 7 are thus identified as good candidates for further treatment regimens directed toward improving mental health.

Additional modifications and improvements of the present invention may also be apparent to those of ordinary skill in the art. Thus, the particular combination of components and steps described and illustrated herein is intended to represent only certain embodiments of the present invention, and is not intended to serve as limitations of alternative devices and methods within the spirit and scope of the invention. Along these lines, it should be understood that the assessment of the quality of life of the patients can be performed by methods other than those specifically described, such as with other types of questionnaires or diagnostic tests. Also, other algorithmic manipulations other than those specifically described may be performed to compare the patient's score to a standard score or to compare the patient's mental and physical health scores to one another. Also, the treatment regimens assigned may take any of a variety of forms that are known or later developed in the art, and further contemplates that existing or newly developed treatment regimens should fall within the scope of the present invention. Also, it should be understood that the method can be performed to improve quality of life of patients suffering from chronic or long-term illnesses or other conditions that are other than those particularly described.

What is claimed is:

1. A method for improving the quality of life of a patient, the method comprising:
(a) assessing the patient's quality of life by evaluating parameters relating to the patient's health, and assigning a score in relation to the evaluated parameters;
(b) comparing the patient's score to a standard score; and
(c) assigning a treatment regimen to the patient on the basis of the comparison obtained in step (b),
whereby the patient's quality of life is improved by receiving the treatment regimen based on the comparison of the patient's score to the standard score.

2. The method of claim 1, wherein step (a) comprises providing at least one health evaluation questionnaire for the patient to complete, the at least one health evaluation questionnaire comprising health questions relating to at least one of physical and mental health parameters, awarding points in relation to answers given by the patient to each health question, and assigning the patient's score by totaling the points awarded for each health question.

3. The method of claim 2, wherein step (a) comprises providing at least one health evaluation questionnaire comprising health questions relating to the patient's own perception of at least one of their physical and mental health.

4. The method of claim 2 wherein step (a) comprises assigning a physical health score to the patient that corresponds to answers to health questions relating to physical health parameters, and assigning a mental health score to the patient that corresponds to answers to health questions relating to mental health parameters.

5. The method of claim 4, wherein step (b) comprises comparing the patient's physical and mental health scores to standard physical and mental health scores that correspond to mean physical and mental health scores obtained from a group of patients that have completed the at least one health evaluation questionnaire.

6. The method of claim 5, wherein step (b) comprises calculating the difference between (i) the patient's mental health score and the standard mental health score, and (ii) the patient's physical health score and the standard physical health score.

7. The method of claim 6, wherein step (b) comprises evaluating a standard deviation of the standard mental health score and a standard deviation of the standard physical health score, and comparing these standard deviations to the calculated differences between (i) the patient's mental health score and standard mental health score, and (ii) patient's physical health score and standard physical health score, to determine whether the calculated differences are significantly above or below the standard mental and physical health scores.

8. The method of claim 7, wherein step (c) comprises assigning a mental health treatment regimen to the patient when the difference between the patient's mental health score and the standard mental health score exceeds the standard deviation of the standard mental health score, and wherein step (c) comprises assigning a physical health treatment regimen to the patient when the difference between the patient's physical health score and the standard physical health score exceeds the standard deviation of the standard physical health score.

9. The method of claim 4 wherein step (b) comprises comparing the patient's physical and mental health scores to standard physical and mental health scores that correspond to a ratings system for evaluating the patient's overall physical and mental health.

10. The method of claim 4 further comprising taking a ratio of the patient's physical health score to the patient's mental health score, and evaluating therefrom which of the patient's physical or mental status is better.

11. The method of claim 10 wherein step (c) comprises assigning a mental health treatment regimen to the patient when the patient's mental health is worse than the patient's physical health, and wherein step (c) comprises assigning a physical health treatment regimen to the patient when the patient's physical health is worse than the patient's mental health.

12. The method of claim 1 wherein step (c) comprises assigning the patient to a disease management program on the basis of the comparison made in step (b).

13. A computer software program operative to implement the method of claim 1, the software program comprising:
(a) assessment program code operative to assess the patient's quality of life by evaluating the parameters relating to the patient's health, and assigning the score in relation to the evaluated parameters;
(b) comparison program code operative to compare the assigned score to the standard score; and
(c) treatment program code operative to assign the treatment regimen to the patient on the basis of the comparison obtained by the comparison software code.

14. A method for improving the quality of life of a patient, the method comprising:
(a) evaluating parameters relating to the patient's mental health, and assigning a mental health score in relation to the evaluated parameters;
(b) evaluating parameters relating to the patient's physical mental health, and assigning a physical health score in relation to the evaluated parameters;
(c) comparing the patient's mental health score to the patient's physical health score; and
(d) assigning a treatment regimen to the patient on the basis of the comparison obtained in step (c),
whereby the patient's quality of life is improved by receiving the treatment regimen based on the comparison of the patient's mental and physical health scores.

15. The method of claim 14, wherein steps (a)-(b) comprise providing at least one health evaluation questionnaire for the patient to complete, the at least one health evaluation questionnaire comprising health questions relating to mental health and physical health parameters, and wherein steps (a)-(b) comprise awarding points in relation to answers given by the patient to each health question, and assigning the patient's mental health score by totaling the points awarded for each health question relating to mental health parameters, and assigning the patient's physical health score by totaling the points awarded for each health question relating to physical health parameters.

16. The method of claim 15, wherein steps (a)-(b) comprises providing at least one health evaluation questionnaire comprising health questions relating to the patient's perception of their own physical and mental health.

17. The method of claim 15 wherein step (c) comprises taking a ratio of the patient's physical health score to the patient's mental health score, and evaluating therefrom which of the patient's physical or mental status is better.

18. The method of claim 15 wherein step (d) comprises assigning a mental health treatment regimen to the patient when the patient's mental health is worse than the patient's physical health, and assigning a physical health treatment regimen to the patient when the patient's physical health is worse than the patient's mental health.

19. The method of claim 15 further comprising step (e) of comparing the patient's physical and mental health scores to standard physical and mental health scores that correspond to a ratings system for evaluating the patient's overall physical and mental health.

20. The method of claim 15, further comprising step (e) of comparing the patient's physical and mental health scores to standard physical and mental health scores that correspond to mean physical and mental health scores obtained from a group of patients that have completed at least one health evaluation survey comprising the health questions relating to at least one of mental and physical health parameters.

21. The method of claim 20, wherein step (e) comprises calculating the difference between (i) the patient's mental health score and the standard mental health score, and (ii) the patient's physical health score and the standard physical health score.

22. The method of claim 21, wherein step (e) comprises evaluating a standard deviation of the standard mental health score and a standard deviation of the standard physical health score, and comparing the calculated differences between (i) the patient's and standard mental health score, and (ii) the patient's and standard physical health score, to the standard deviations, to determine whether the calculated differences are significantly above or below the standard mental and physical health scores.

23. The method of claim 22, wherein step (d) comprises assigning a mental health treatment regimen to the patient when the difference between the patient's mental health score and the standard mental health score exceeds the standard deviation of the standard mental health score, and wherein step (d) comprises assigning a physical health treatment regimen to the patient when the difference between the patient's physical health score and the standard physical health score exceeds the standard deviation of the standard physical health score.

24. The method of claim 14 wherein step (d) comprises assigning the patient to a disease management program on the basis of the comparison made in step (c).

25. A computer software program operative to implement the method of claim 14, the software program comprising:
(a) assessment program code operative to assess the patient's quality of life by:
i. evaluating the parameters relating to the patient's mental health, and assigning the mental health score in relation to the evaluated parameters; and
ii evaluating parameters relating to the patient's physical health, and assigning the physical health score in relation to the evaluated parameters;
(b) comparison program code operative to compare the mental health score to the physical health score; and
(c) treatment program code operative to assign the treatment regimen to the patient on the basis of the comparison obtained by the comparison software code.
